(19) United States
(12) Patent Application Publication
(10) Pub. No.: US 2015/0127043 A1
(43) Pub. Date: May 7, 2015

(54) Balloon Catheter Having a Pre-Positioned Refold Tool

(71) Applicant: Medtronic Vascular, Inc., Santa Rosa, CA (US)

(72) Inventor: Jonathan Cope, Galway City (IE)

(73) Assignee: Medtronic Vascular, Inc., Santa Rosa, CA (US)

(21) Appl. No.: 14/074,109

(22) Filed: Nov. 7, 2013

(51) Int. Cl. A61M 25/10

(52) U.S. Cl. CPC A61M 25/104
USPC 606/194

(57) ABSTRACT

A balloon catheter includes an elongated catheter shaft, a hub disposed at a proximal portion of the catheter shaft, an inflatable balloon disposed at a distal portion of the catheter shaft, and a refold tool slidingly disposed over the elongated catheter shaft and positioned between the hub and the inflatable balloon. The balloon is configured to transform between an inflated configuration and a deflated configuration, the balloon having a deflated profile when in the deflated configuration. The refold tool defines a lumen there-through that is flared at a distal end thereof, and the lumen of the tubular component of the refold tool is sized to refold the balloon to a refolded profile that is smaller than the deflated profile when the balloon in the deflated configuration passes through the refold tool.

### FIELD OF THE INVENTION

The invention in general relates to balloon catheters, and in particular relates to refold tools for refolding balloons of balloon catheters,

### BACKGROUND OF THE INVENTION

Balloon angioplasty catheters are widely used in angioplasty procedures for efficiently and effectively opening stenoses in the coronary arteries and in other parts of the vascular system. The most widely used form of angioplasty makes use of a dilatation catheter which has an inflatable balloon at its distal end. Using fluoroscopy, the physician guides the catheter through the vascular system until the deflated balloon is positioned across the stenosis. The balloon is then inflated by supplying a fluid under pressure through an inflation lumen to the balloon. The inflation of the balloon causes stretching of the artery and pressing of the lesion into the artery wall to re-establish acceptable blood flow through the artery.

One important characteristic of a dilatation catheter used for angioplasty is its profile, i.e., the outer diameter or dimension of the distal end portion thereof The profile dimension affects the ease and ability of the dilatation catheter to cross a narrowing or stenosis. Considerable effort has been spent in developing low-profile dilatation balloon catheters by minimizing the dimensions of the core or inner tube which extends through the balloon to its distal end, and by reducing wall thickness, to the extent possible, of the balloon itself.

In the deflated state, an inelastic angioplasty balloon may have flaps or wings that must be folded or wrapped around the balloon catheter to achieve a low crossing profile and to assist in getting the balloon catheter to fit within and pass through the guide catheter lumen. In some instances, it is necessary to rewrap a balloon prior to use in situ within a patient. For example, a deflated balloon may need to be refolded after inflation to purge air from the catheter and balloon prior to use in the patient. in another example, the balloon may be shipped deflated but not folded or wrapped within packaging. This may be desirable because storing a balloon in a wrapped or folded configuration may cause the folds or wings thereof to stick together, resulting in tearing or ripping of the balloon when inflated. Thus, if the balloon is shipped in a deflated configuration but is not wrapped or folded, the deflated balloon needs to be folded to a low profile to achieve the smallest or lowest profile possible for delivery into a patient. In yet another example, a balloon may need to be refolded if it has been used in the patient to dilate a stenosis. If the balloon catheter has been removed from the patient and the clinician wishes to reinsert the catheter, e.g. to dilate a different stenosis or redilate the same stenosis, then it is useful to refold the balloon. In all of these applications, the deflated balloon needs to be refolded to a refolded low profile for delivery.

Thus, it has been customary to provide a refold or rewrap tool kit with the balloon angioplasty catheters that the physician employs to tightly wind the balloon flaps around one another. These kits are typically shipped within a pouch that is attached to the catheter packaging, and include the Sci-Med® Wrap-It™ refolding tool included with the Sci-Med® NC Bandit™ PTCA catheter, the CDV™ balloon refolding tool sold with the CVD FACT™ PTCA catheter distributed by the assignee of the present invention, and the ACS® balloon sheath. An exemplary conventional refold tool 103 and method of use is shown in FIGS. 1-2. In order to refold a deflated balloon 101, refold tool 103 is initially advanced over a distal end of balloon 101 in a proximal direction as shown with directional arrow 105 in FIG. 1. Especially with respect to larger balloons, feeding the distal end of the balloon into the refold tool may be quite difficult and may lead to bunching of the proximal portion of the balloon as shown in FIG. 2. Further, forcing the refold tool 103 over the balloon 101 in a proximal direction places a longitudinal compression force on the catheter 100. Such a longitudinal compression force may result in buckling and/or kinking in the catheter shaft. In some instances, a stiffener needs to be inserted into the catheter to prevent such buckling/kinking.

Accordingly, there is a continuing need for improved balloon rewrapping or refolding tools and methods for rewrapping balloon flaps of medical balloon catheters.

### BRIEF SUMMARY OF THE INVENTION

Embodiments hereof relate to a balloon catheter including an elongated catheter shaft, a hub disposed at a proximal portion of the catheter shaft, an inflatable balloon disposed at a distal portion of the catheter shaft, and a refold tool slidingly disposed over the elongated catheter shaft and pre-positioned between the hub and the inflatable balloon. The inelastic balloon is configured to transform between an inflated configuration and a deflated configuration having wings or flaps, the balloon having a deflated profile when in the deflated configuration. The refold tool defines a lumen there-through that is flared at a distal end thereof, and the lumen of the tubular component of the refold tool is sized to refold the balloon to a refolded profile that is smaller than the deflated profile when the balloon in the deflated configuration passes through the refold tool.

Embodiments hereof also relate to a method of refolding a deflated balloon of a balloon catheter following an inflation of the balloon. The balloon catheter includes an elongated catheter shaft, a hub disposed at a proximal portion of the catheter shaft, the balloon disposed at a distal portion of the catheter shaft, and a refold tool slidingly disposed over the elongated catheter shaft and initially positioned between the hub and the balloon. The refold tool defines a lumen there-through that is flared at a distal end thereof. In order to refold the balloon, the refold tool is distally advanced over the catheter shaft towards the balloon until the flared portion of the lumen of the refold tool is positioned adjacent to and proximal to a proximal end of the balloon. The balloon is in a deflated configuration and has a deflated profile. The catheter shaft and the refold tool are moved relative to each other such that the deflated balloon passes through the lumen of the refold tool, whereby the deflated balloon is refolded to a refolded profile that is smaller than the deflated profile.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIGS. 1-2 illustrate a prior art refold tool and method of use thereof.

FIG. 3 is a side view of a balloon catheter having a pre-positioned refold tool thereon according to an embodiment hereof, wherein a balloon of the balloon catheter is shown in an inflated configuration.

FIG. 3A is a cross-sectional view taken along line A-A of FIG. 3.

FIG. 3B is a cross-sectional view taken along line A-A of FIG. 3 according to an alternative embodiment hereof in which the multi-lumen balloon catheter shaft is formed via extrusion.

FIG. 4 is a perspective view of the refold tool of FIG. 3 removed from the balloon catheter for illustrative purposes only.

FIG. 4A is a sectional view taken along line A-A of FIG. 4.

FIG. 5 is a sectional view of a refold tool according to an alternative embodiment hereof.

FIG. 6 is an enlarged side view of balloon catheter and pre-positioned refold tool of FIG. 3, wherein the refold tool is pre-attached to a hub of the balloon catheter.

FIG. 7 is an enlarged side view of a balloon catheter and a pre-positioned refold tool according to another embodiment hereof, wherein the refold tool is pre-attached to a hub of the balloon catheter with a plurality of perforations.

FIG. 8 is a side view of a balloon catheter and a pre-positioned refold tool according to another embodiment hereof, wherein the refold tool is pre-mounted onto the balloon catheter but not pre-attached to a hub thereof.

FIGS. 9-12 illustrate a method of use of the balloon catheter and refold tool of FIG. 3.

FIGS. 13-14 illustrate a refold tool according to another embodiment hereof and method of use thereof.

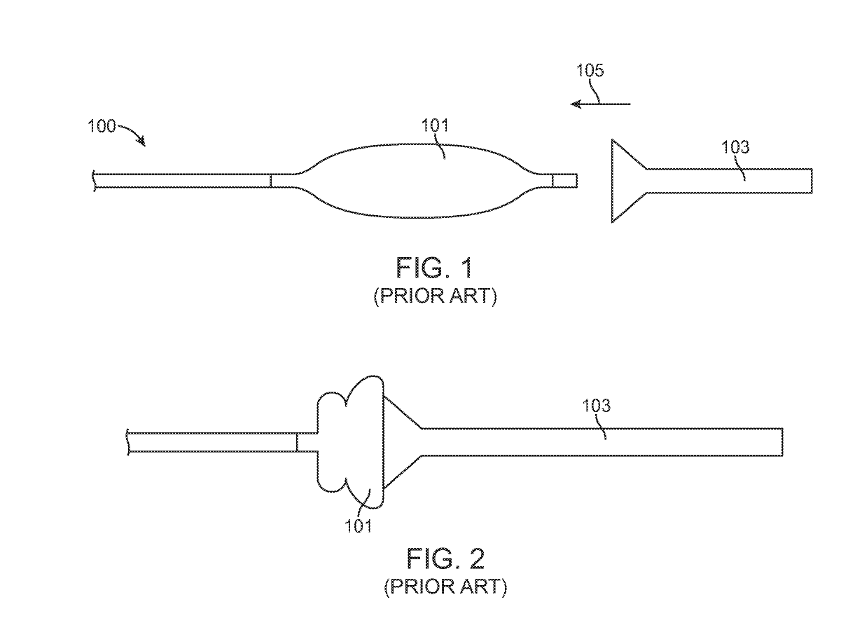

### DETAILED DESCRIPTION OF THE INVENTION

Specific embodiments of the present invention are now described with reference to the figures, wherein like reference numbers indicate identical or functionally similar elements. The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description. The terms “distal” and “proximal” are used in the following description with respect to a position or direction relative to the treating clinician. “Distal” and “distally” are positions distant from or in a direction away from the clinician. “Proximal” and “proximally” are positions near or in a direction toward the clinician.

Embodiments hereof relate to a balloon catheter having a proximal hub, an elongated catheter shaft, a distal balloon, and a refold tool pre-positioned or pre-disposed between the proximal hub and the distal balloon so that the refold tool can be advanced in a distal direction and initially applied to a proximal end of the balloon. In this manner, the elongated catheter shaft is under longitudinal tension as the refold tool is distally advanced over the balloon and therefore allows the balloon to be advanced through the refold tool with ease. More particularly, undesired bunching and subsequent damage of the balloon and/or the catheter shaft is prevented. In addition, since the elongated catheter shaft is under longitudinal tension as the refold tool is distally advanced over the balloon, the use of a mandrel or other stiffening member is not required for structural support during balloon refolding, thereby saving the user both time and effort. As used herein, the terms “pre-disposed” and “pre-positioned” refer to the initial position or location of the refold tool prior to use of the refold tool to fold or wrap a deflated balloon. Pre-positioned refold tools include refold tools described herein that are pre-attached or detachably coupled to the proximal hub as well as refold tools described herein that are pre-mounted between the proximal hub and the distal balloon but not attached or coupled to the proximal hub.

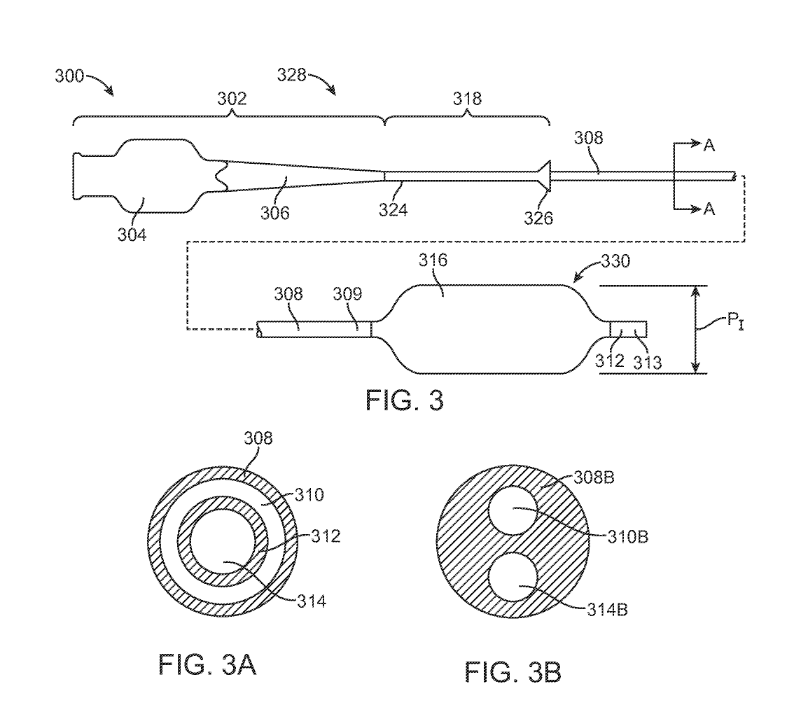

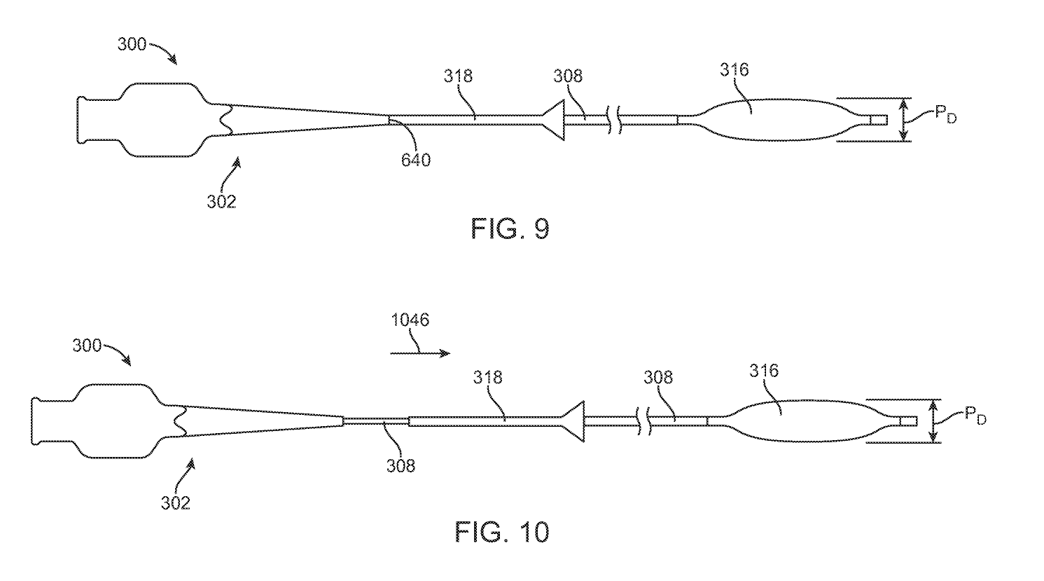

More particularly, a side view of a balloon catheter 300 according to an embodiment hereof is shown in FIG. 3. Balloon catheter 300 includes a proximal portion 328 that extends out of the patient during clinical use and has a hub 302. As would be understood by one of ordinary skill in the art of balloon catheter design, hub 302 includes a luer fitting 304 or other type of fitting that may be connected to a source of inflation fluid and may be of another construction or configuration without departing from the scope of the present invention. Hub 302 may also include a strain relief segment 306 coupled to luer 304. A distal portion 330 of catheter 300 is positionable at a target location within the vasculature and includes an inflatable balloon 316, which is shown in an expanded or inflated configuration having an inflated diameter or profile P_{I} in FIG. 3. As used herein, the term “profile” is intended to refer to a transverse dimension of a structure or component, such as a medical balloon or catheter shaft. If the component has a circular cross-section, the transverse dimension is an outer diameter thereof. In the inflated configuration, inflated profile P_{I} may range between 1.50 mm to 4.0 mm for use in coronary arteries, but the invention is not on limited. As will be described in more detail herein, balloon 316 is configured to transform between the inflated configuration and a deflated configuration. When in the deflated configuration, balloon 316 forms a wing, a flap or a plurality of flaps as will be described in more detail herein. In the deflated configuration, balloon 316 has a deflated profile P_{D} which is shown and described with respect to FIG. 9 and may be measured from “wingtip” to “wingtip.” However, when refolded, balloon 316 has a refolded profile P_{R} which is shown and described with respect to FIG. 12 and, for coronary artery dilatation balloons, may range between 0.75 mm to 1.35 mm. In embodiments hereof, catheter 300 may be used in balloon angioplasty procedures, as well as may form the basis of a stent delivery system, a graft delivery system, and/or a drug delivery system.

In one embodiment illustrated in FIG. 3A, balloon catheter 300 has an over-the-wire (OTW) coaxial catheter configuration with an outer tubular component or shaft 308 and an inner shaft 312. Outer shaft 308 has a proximal end (obscured from view in FIG. 3) coupled to hub 302 and a distal end 309 coupled to a proximal end of balloon 316. Inner shaft 312 defines a guidewire lumen 314 extending substantially the entire length of the catheter for accommodating a guidewire (not shown). Inner shaft 312 has a proximal end (obscured from view in FIG. 3) coupled to hub 302 and a distal end 313 terminating distally of balloon 316 and defining a distal guidewire port. Inner shaft 312 extends coaxially within outer shaft 308 such that an annular inflation lumen 310 is defined between an inner surface of outer shaft 308 and an outer surface of inner shaft 312. Inflation lumen 310 thus extends within outer catheter shaft 308 and into the inner volume of balloon 316 to allow inflation fluid received through an inflation port of luer fitting 304 to be delivered to balloon 316. A distal end of balloon 316 is coupled to inner shaft 312.

Other types of catheter construction are also amendable to the invention, such as, without limitation thereto, a catheter shaft formed by multi-lumen profile extrusion. For example, an alternate catheter construction is illustrated in FIG. 3B. Rather than including coaxial inner and outer catheter shafts, a single catheter shaft 308B may define an inflation lumen 310B and a guidewire lumen 314B, each extending substantially the entire length of the catheter and parallel to each other. Although depicted as circular in cross-section, one or more lumen(s) of shaft 308B may have any suitable cross-section including for example circular, elliptical, or crescent shapes. In addition, balloon catheter 300 may have a rapid-exchange configuration with the guidewire lumen extending only along a distal portion of the catheter, as understood by those of ordinary skill in the art. Regardless of the type of catheter construction, the catheter shaft(s) may be formed of a polymeric material, non-exhaustive examples of which include polyethylene, polyethylene block amide (PEBA), polyamide and/or combinations thereof. Optionally, the catheter shaft or some portion thereof may he formed as a composite having a reinforcement material incorporated within a polymeric body in order to enhance strength and/or flexibility. Suitable reinforcement layers include braiding, wire mesh layers, embedded axial wires, embedded helical or circumferential wires, and the like. In one embodiment, for example, at least a proximal portion of outer catheter shaft 308 may be formed from a reinforced polymeric tube.

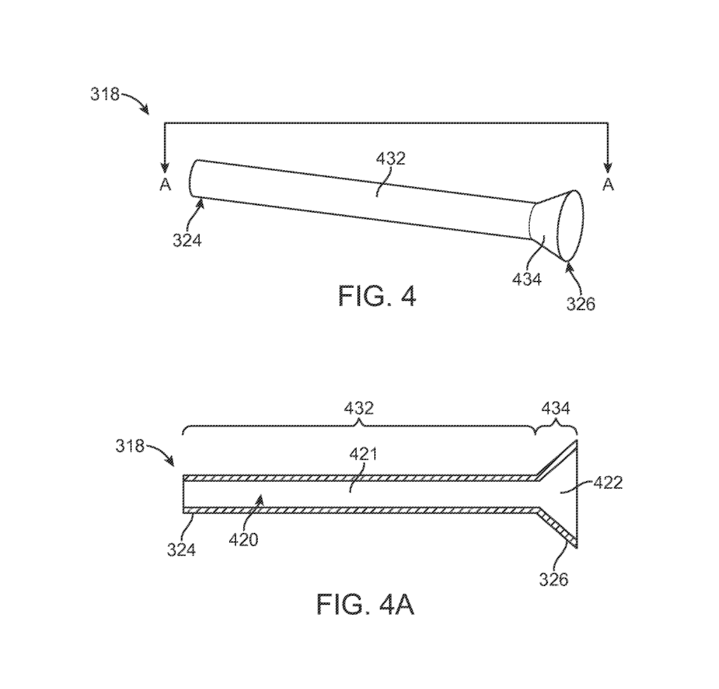

Balloon catheter 300 includes a refold tool 318 slidingly disposed over outer shaft 308 and initially positioned or pre-positioned between hub 302 and balloon 316. With additional reference to the perspective and sectional views of FIGS. 4 and 4A, respectively, refold tool 318 is a tabular body or component that defines a continuous lumen 420 extending the entire length thereof between a port at proximal end 324 to a port at distal end 326. Lumen 420 is flared or funnel shaped at a distal portion 422 thereof. In an embodiment hereof, refold tool 318 includes a cylindrical component or portion 432 and a frustoconical component or portion 434, the frustoconical component defining the flared distal portion 422 of lumen 420. More particularly, as shown in the sectional view of FIG. 4A, lumen 420 includes a cylindrical proximal portion 421 of a substantially constant diameter along cylindrical component 432 and flared distal portion 422 of increasing dimension in a distal direction along frustoconical component 434. It will be understood by one of ordinary skill in the art that portions 421, 422 form continuous lumen 420 through refold tool 318 but are described with separate reference numbers for clarity. Distal portion 422 of lumen 420 is flared to permit entry of or to guide deflated balloon 316 into refold tool 318, while proximal portion 421 of lumen 420 is sized to refold balloon 316 to a refolded profile that is smaller than the deflated profile when the deflated balloon passes through the refold tool. Stated another way, refold tool 318 is configured to refold balloon 316 when the deflated balloon is pulled through the refold tool in a proximal direction or refold tool 318 is advanced distally over balloon 316.

In an embodiment, cylindrical component 432 and a frustoconical component 434 are integrally formed from a single or unitary piece of material such as polyethylene, polypropylene or other polyolefins, or fluorinated polymers such as fluorinated ethylene propylene (FEP) or polytetrafluoroethylene (PTFE). Low-friction materials may be selected to facilitate sliding refold tool 318 with respect to balloon 316 and catheter shaft 308. For example, cylindrical component 432 and frustoconical component 434 may be formed as a single molded component. In another embodiment, cylindrical component 432 and frustoconical component 434 may be two or more separate components of the same or different materials that are coupled together via heat bonding or any suitable mechanical method such as via an adhesive.

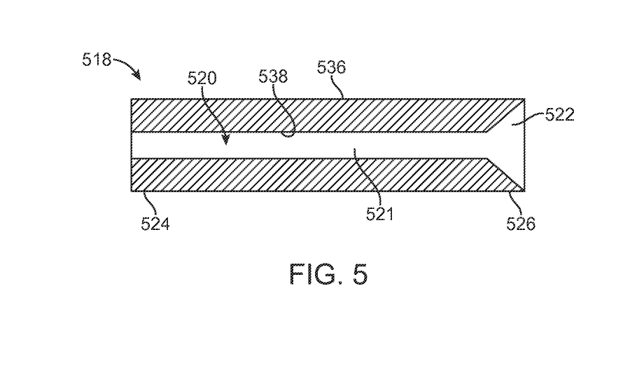

Other types of construction for refold tool 318 are also amendable to the invention, such as, without limitation thereto, the configuration of a refold tool 518 shown in FIG. 5. Similar to refold tool 318, refold tool 518 is a tubular body or component that defines a continuous lumen 520 extending the entire length thereof between a proximal end or port 524 to a distal end or port 526. Lumen 520 has a cylindrical proximal portion 521 and a flared distal portion 522. However, in this embodiment, refold tool 5018 has an outer surface 536 of a constant profile or diameter, while an inner surface 538 defines the shape of lumen 520 such that distal portion 522 of the lumen is flared or funnel shaped. Although the configuration of the respective refold tools differ, lumen 520 of refold tool 518 is shaped similar to lumen 420 of refold tool 318 such that the distal flared portions thereof permit entry of deflated balloon 316 into the refold tool while the proximal cylindrical portions thereof are sized to refold balloon 316 as described above.

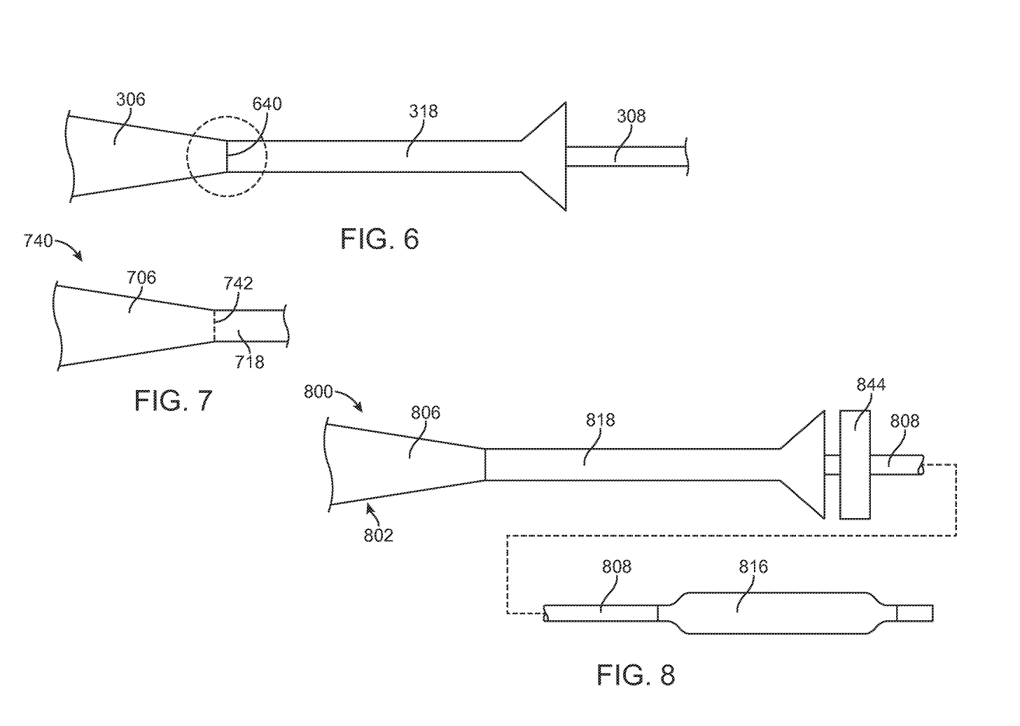

In an embodiment hereof, refold tool 318 is pre-attached or detachably coupled to hub 302 of balloon catheter 300 to secure the refold tool in place prior to use. More particularly, with additional reference to FIG. 6, refold tool 318 is assembled or manufactured as pre-attached to strain relief 306 of hub 302 via a breakable connection or joint 640. Joint 640 is breakable such that refold tool 318 may be detached or separated from hub 302 and slidingly distally advanced over outer shaft 308 and balloon 316 in order to refold the balloon. Breakable joint 640 may be broken manually via twisting or pulling such that refold tool 318 may be detached or separated from hub 302 without the use of a tool. Stated another way, breakable joint 640 is configured such that refold tool 318 may be separated or broken apart from hub 302 via application of reasonable manual torque or force without damage to the refold tool or to the hub of the catheter.

There are several possible ways to construct breakable joint 640 such that refold tool 318 is manually detachable from hub 302. In an embodiment hereof, breakable joint 640 may include a low-strength adhesive. In another embodiment, breakable joint 640 may be formed by overmolding refold tool 318 onto a distal end of strain relief 306 of hub 302 with a low-strength material or the same material, but thinner. In another embodiment hereof, breakable joint 640 may be formed by disposing refold tool 318 onto a distal end of strain relief 306 of hub 302 with a tight interference or friction fit. In yet another embodiment, depicted in FIG. 7, a breakable joint 740 according to another embodiment hereof may include a higher-strength adhesive or overmolded material that includes an area of weakness 742 there-through such that a refold tool 718 may be separated or broken apart from a strain relief 706 via manual force. Area of weakness 742 may include one or more perforations, slots, slits, or grooves formed through the material of refold tool 718. Regardless of how the breakable joint is formed, the refold tool is securely attached onto the strain relief and remains coupled to the catheter hub until the refold tool is required for use, at which point the refold tool may be decoupled or separated from the hub and utilized to refold the catheter balloon.

While it is not required that the refold tool be pre-attached or detachably coupled to the catheter hub, the refold tool is pre-mounted or pre-disposed on the catheter, distal to the hub and proximal to the balloon. For example, FIG. 8 illustrates another embodiment hereof in which a refold tool 818 is pre-mounted onto a catheter 800 having a catheter shaft 808 and a distal balloon 816. Refold tool 818 is slidingly disposed over catheter shaft 808, adjacent to or abutting against strain relief 806 of hub 802 at the proximal portion of the catheter. In this embodiment, refold tool 818 is secured or held in position via a removable stopper 844 but is not attached or coupled to hub 802. Removable stopper 844 prevents refold tool 818 from sliding over the catheter and/or off the catheter prior to use. When it is desired to use refold tool 818, stopper 844 may be ripped, cut, or otherwise removed from the catheter such that the refold tool may be distally advanced towards balloon 816.

A method of refolding balloon 316 of balloon catheter 300 via refold tool 318 will now be described with reference to FIGS. 9-12. FIG. 9 illustrates balloon catheter 300 having refold tool 318 pre-attached to hub 302 as described above with respect to FIGS. 3 and 6, with balloon 316 being shown in a deflated configuration having wings defining a deflated profile P_{D}. Deflated balloon 316 may have been previously inflated for purging reasons or may have been stored and shipped in the deflated configuration, as described in more detail in the background section above. When it is desired to fold or wrap the deflated balloon into a refolded profile for delivery, refold tool 318 is first detached or decoupled from hub 302 as shown in FIG. 10. Refold tool 318 is detached from hub 302 by breaking breakable joint 640 as described above with respect to FIG. 6 and is advanced in a distal direction indicated by directional arrow 1046 towards balloon 316. In one embodiment, breaking low-strength joint 640 includes applying torque to refold tool 318 or pulling refold tool 318 and hub 302 in opposite directions,

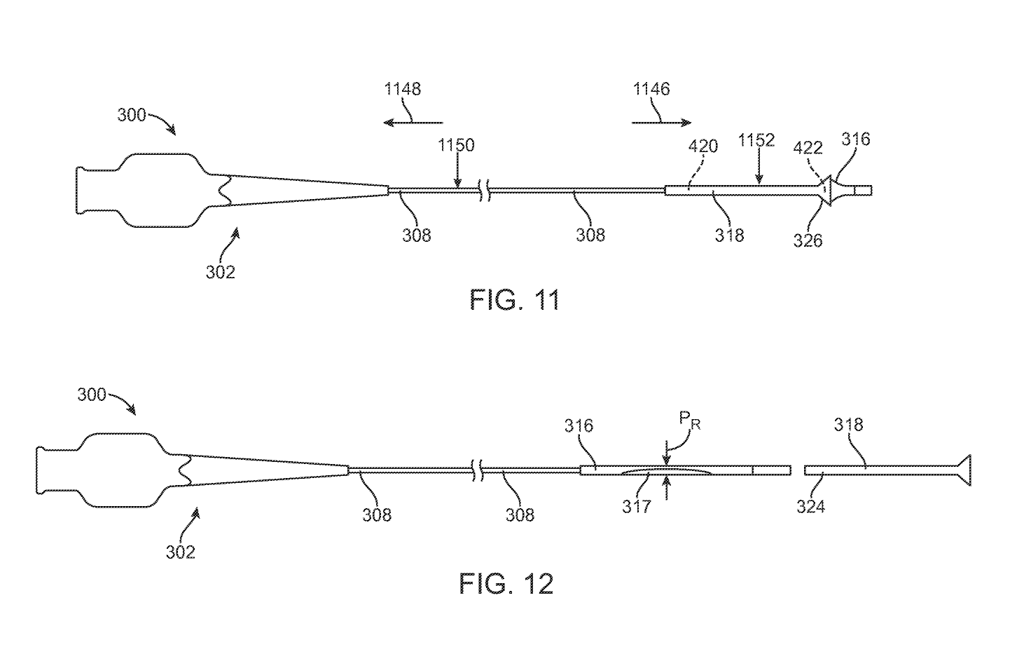

After refold tool 318 is separated from hub 302, the refold tool is then distally advanced over catheter shaft 308 towards deflated balloon 316 as shown in FIG. 11. Catheter shaft 308 and refold tool 318 are then moved relative to each other until deflated balloon 316 is radially compressed or constrained within lumen 420 of refold tool 318. Moving catheter shaft 308 and refold tool 318 relative to each other may include retracting catheter shaft 308 in a proximal direction indicated by directional arrow 1148 while holding refold tool 318 stationary, or by advancing refold tool 318 in a distal direction indicated by directional arrow 1146 while holding catheter shaft 308 stationary, or a combination of proximally retracting catheter shaft 308 and distally advancing refold tool 318. While moving catheter shaft 308 and refold tool 318 relative to each other, a user grasps catheter shaft 308 at a first position or location proximal to refold tool 318, represented by arrow 1150, with one hand and grasps refold tool 318 at a second position or location, represented by arrow 1152, with the other hand. When deflated balloon 316 is being advanced through lumen 420 of refold tool 318, the user's hands move apart or in opposing directions. Catheter shaft 308 is under longitudinal tension during the step of moving the catheter shaft and the refold tool relative to each other, and as such, buckling or kinking of catheter shaft 308 is prevented as the balloon material is fed through the refold tool. in addition, since catheter shaft 308 is under longitudinal tension during the refolding process, it is not required to have a mandrel or other stiffening component disposed through the catheter shaft to prevent lateral deflection that could result in buckling or kinking.

Refold tool 318 may be distally advanced until balloon 316 has made it through the refold tool, such that proximal end 324 of refold tool 318 is located distal to the distal end of balloon 316 and balloon 316 is thereby refolded to a refolded profile P_{R} that is smaller than the deflated profile P_{D}. When in the refolded profile P_{R}, wings of balloon 316 are snugly wrapped around the deflated balloon and against inner shaft 312. If desired, refold tool 318 is distally advanced until the refold tool is separated or removed from catheter 300 as shown in FIG. 12. After removal from catheter 300, refold tool 318 may be discarded.

In another embodiment hereof, it may be desirable to reposition the refold tool onto the balloon catheter after initial use. For example, once the balloon has been refolded inside refold tool 318, the tool may be slid proximally to revert to its original location between the hub and the inflatable balloon. This cycle can be repeated if it is desirable to pass a deflated balloon into the refold tool several times to ensure that the balloon is refolded to the smallest possible profile for delivery, or it may be desirable to refold a deflated balloon after use or inflation in situ such as between successive inflations within the same patient.

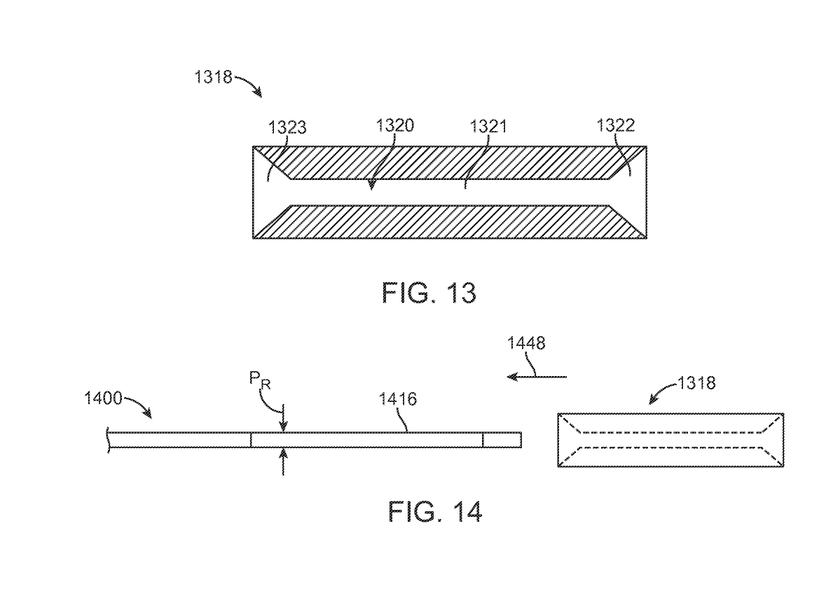

FIG. 13 illustrates a side sectional view of a refold tool 1318 according to another embodiment hereof Although shown removed from a balloon catheter in FIG. 13, refold tool 1318 is pre-positioned onto a balloon catheter 1400 (see FIG. 14) between a proximal hub (not shown) and a distal balloon 1416 (see FIG. 14) as described above with respect to refold tool 318 of FIG. 3. However, refold tool 1318 is a tubular body or component that defines a continuous lumen 132.0 that is flared at a distal portion 1322 and a proximal portion 1323 thereof, with a straight intermediate portion 1321 between the flared distal and proximal portions that is sized to refold a balloon. It will be understood by one of ordinary skill in the art that portions 1321, 1322, 1323 form continuous lumen 1320 through refold tool 1318 but are described with separate reference numbers for clarity. Proximal portion 1323 of lumen 1320 is flared to permit entry of or to guide a distal end of deflated balloon 1416 into refold tool 1318 if it is desired to reposition a removed refold tool onto balloon catheter 1400 after initial use. When advanced in a proximal direction indicated by directional arrow 1448 over a distal end of deflated balloon 1416, a stylet or other stiffening member (not shown) may be disposed in lumen 314 of balloon catheter 1400 in order to prevent buckling of the catheter. If desired, refold tool 1318 may be re-sterilized prior to being repositioned onto balloon catheter 1400. Although. reuse and repositioning of a refold tool is described with respect to refold tool 1318 having flared distal and proximal portions 1322, 1323, a pre-positioned refold tool having any configuration described herein may be repositioned onto a balloon catheter after initial use. If the refold tool includes a lumen having only a flared distal portion, such as refold tool 318 or refold tool 518, the refold tool may be flipped around after removal from the balloon catheter and the flared distal portion can be applied over the balloon in a distal to proximal direction.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. it will also be understood that each feature of each embodiment discussed herein, and of each reference cited herein, can be used in combination with the features of any other embodiment. All patents and publications discussed herein are incorporated by reference herein in their entirety.

What is claimed is:

1. A balloon catheter comprising:
an elongated catheter shaft;
a hub disposed at a proximal portion of the catheter shaft;
an inflatable balloon disposed at a distal portion of the catheter shaft, wherein the balloon is configured to transform between an inflated configuration and a deflated configuration, the balloon having a deflated profile when in the deflated configuration; and
a refold tool slidingly disposed over the elongated catheter shaft and pre-positioned between the hub and the inflatable balloon, the refold tool defining a lumen there-through that is flared at a distal end thereof, wherein the lumen of the tubular component of the refold tool is sized to refold the balloon to a refolded profile that is smaller than the deflated profile when the balloon in the deflated configuration passes through the refold tool.

2. The balloon catheter of claim 1, wherein the refold tool is detachably coupled to a distal end of the hub.

3. The balloon catheter of claim 2, wherein the refold tool is overmolded onto the distal end of the hub with a low strength connection that is manually breakable to detach the refold tool from the distal end of the hub.

4. The balloon catheter of claim 2, wherein the refold tool is detachably coupled to the distal end of the hub via an interference fit.

5. The balloon catheter of claim 1, wherein the refold tool includes a cylindrical component and a frustoconical component coupled at a distal end of the cylindrical component, the frustoconical component defining the flared portion of the lumen of the refold tool.

6. The balloon catheter of claim 1, wherein the refold tool includes a tubular component having a constant outer surface and a varied inner surface, the varied inner surface defining the flared portion of the lumen of the refold tool.

7. The balloon catheter of claim 1, wherein the hub includes a luer and a strain relief segment coupled to a distal end of the luer.

8. A balloon catheter comprising:
an elongated catheter shaft;
a hub disposed at a proximal portion of the catheter shaft;
an inflatable balloon disposed at a distal portion of the catheter shaft, wherein the balloon is configured to transform between an inflated configuration and a deflated configuration, the balloon having a deflated profile when in the deflated configuration; and
a refold tool slidingly disposed over the elongated catheter shaft and pre-positioned between the hub and the inflatable balloon, the refold tool defining a lumen there-through that is flared at a distal end thereof, wherein the refold tool is configured to refold the balloon to a refolded profile that is smaller than the deflated profile when the balloon in the deflated configuration is pulled through the refold tool in a proximal direction.

9. The balloon catheter of claim 8, wherein the refold tool is detachably coupled to a distal end of the hub.

10. The balloon catheter of claim 8, wherein the refold tool includes a cylindrical component and a frustoconical component coupled at a distal end of the cylindrical component, the frustoconical component defining the flared portion of the lumen of the refold tool.

11. The balloon catheter of claim 8, wherein the refold tool includes a tubular component having a constant outer surface and a varied inner surface, the varied inner surface defining the flared portion of the lumen of the refold tool.

12. The balloon catheter of claim 8, wherein the hub includes a luer and a strain relief segment coupled to a distal end of the luer.

13. A method of refolding a deflated balloon of a balloon catheter following inflation of the balloon, the balloon catheter having an elongated catheter shaft, a hub disposed at a proximal portion of the catheter shaft, the balloon disposed at a distal portion of the catheter shaft, and a refold tool slidingly disposed over the elongated catheter shaft and initially positioned between the hub and the balloon, wherein the refold tool defines a lumen there-through that is flared at a distal end thereof the method comprising the steps of
distally advancing the refold tool over the catheter shaft towards the balloon until the flared portion of the lumen of the refold tool is positioned adjacent to and proximal to a proximal end of the balloon, wherein the balloon is in a deflated configuration and has a deflated profile;
moving the catheter shaft and the refold tool relative to each other such that the deflated balloon passes through the lumen of the refold tool, whereby the deflated balloon is refolded to a refolded profile that is smaller than the deflated profile.

14. The method of claim 13, wherein there is no stiffening component inserted through the catheter shaft during the step of moving the catheter shaft and the refold tool relative to each other.

15. The method of claim 13, wherein the step of moving the catheter shaft and the refold tool relative to each other includes proximally retracting the catheter shaft while holding the refold tool stationary.

16. The method of claim 13, wherein the step of moving the catheter shaft and the refold tool relative to each other includes distally advancing the refold tool while holding the catheter shaft stationary.

17. The method of claim 13, wherein the refold tool is detachably coupled to a distal end of the hub and the method further includes the step of detaching the refold tool from the hub prior to the step of distally advancing the refold tool.

18. The method of claim 17, wherein the step of detaching the refold tool from the hub includes applying torque to the refold tool.

19. The method of claim 13, wherein the catheter shaft is under longitudinal tension during the step of moving the catheter shaft and the refold tool relative to each other.

20. The method of claim 13, further comprising the step of:
distally advancing the refold tool until the refold tool is removed from the catheter shaft, wherein the step of distally advancing the refold tool occurs after the step of moving the catheter shaft and the refold tool relative to each other.
